(19) United States
(12) Patent Application Publication
(10) Pub. No.: US 2022/0160415 A1
(43) Pub. Date: May 26, 2022

(54) ELECTROSURGICAL DEVICES AND SYSTEMS HAVING ONE OR MORE POROUS ELECTRODES

(71) Applicant: Apyx Medical Corporation, Clearwater, FL (US)

(72) Inventor: Shawn D. Roman, Safety Harbor, FL (US)

(21) Appl. No.: 17/425,782

(22) PCT Filed: Jan. 27, 2020
(86) PCT No.: PCT/US20/15208

§ 371 Date: Jul. 26, 2021

Related U.S. Application Data
(60) Provisional application No. 62/797,867, filed on Jan. 28, 2019.

(51) Int. Cl. A61B 18/04; A61B 18/14; H05H 1/46

(52) U.S. Cl. CPC A61B 18/042; A61B 2018/00065; H05H 1/4645; A61B 18/14

(57) ABSTRACT

Electrosurgical devices and systems having one or more porous electrodes are provided. An electrosurgical apparatus is provided having a shaft, a handle, and at least one porous electrode. The shaft is coupled to the handle and the at least one porous electrode is disposed on a distal tip of the shaft. The at least one porous electrode conducts energy provided to the distal tip and enables fluid provided to the distal tip to pass through the porous structure of the at least one electrode, such that the electrosurgical energy and the fluid are simultaneously applied to patient tissue adjacent to the at least one porous electrode. In one aspect, the electrosurgical apparatus includes a switching means configured to enable a user to select which of at least a first fluid, e.g. saline, or a second fluid, e.g., helium, to be provided to the distal tip.

### PRIORITY

This application claims priority to U.S. Provisional Patent Application Ser. No. 62/797,867, filed Jan. 28, 2019, entitled “ELECTROSURGICAL DEVICES AND SYSTEMS HAVING ONE OR MORE POROUS ELECTRODES”, the contents of which are hereby incorporated by reference in its entirety.

### BACKGROUND

### Field

The present disclosure relates generally to electrosurgery and electrosurgical systems and apparatuses, and more particularly, to electrosurgical devices and systems having one or more porous electrodes.

### Description of the Related Art

Today, electrosurgery is one of the widely used surgical modalities for treating tissue abnormalities. Electrosurgical devices fall into one of two categories: monopolar devices and bipolar devices. Generally, surgeons are trained in the use of both monopolar and bipolar electrosurgical techniques, and essentially all operating rooms will be found equipped with the somewhat ubiquitous instrumentality for performing electrosurgery.

Monopolar electrosurgical devices typically comprise an electrosurgical probe, or handpiece, having a first or “active” electrode extending from one end. The electrosurgical probe is electrically coupled to an electrosurgical generator, which provides a high frequency electric current. A remote control switch is attached to the generator and commonly extends to a foot switch located in proximity to the operating theater. During an operation, a second or “return” electrode, having a much larger surface area than the active electrode, is positioned in contact with the skin of the patient. The surgeon may then bring the active electrode in close proximity to the tissue and activate the foot control switch, which causes electrical current to arc from the distal portion of the active electrode and flow through tissue to the larger return electrode.

For the bipolar modality, no return electrode is used. Instead, a second electrode is closely positioned adjacent to the first electrode, with both electrodes being attached to an electrosurgical probe, or handpiece. As with monopolar devices, the electrosurgical probe is electrically coupled to an electrosurgical generator. When the generator is activated, electrical current arcs from the end of the first electrode to the end of the second electrode, flowing through the intervening tissue. In practice, several electrodes may be employed, and depending on the relative size or locality of the electrodes, one or more electrodes may be active.

Whether arranged in a monopolar or bipolar fashion, the active electrode may be operated to either cut tissue or coagulate tissue. When used to cut tissue, the electrical arcing and corresponding current flow results in a highly intense, but localized heating, sufficient enough to break intercellular bonds, resulting in tissue severance. When used to coagulate tissue, the electrical arcing results in a low level current that denatures cells to a sufficient depth without breaking intercellular bonds, i.e., without cutting the tissue.

Whether tissue is cut or coagulated mainly depends on the geometry of the active electrode and the nature of the electrical energy delivered to the electrode. In general, the smaller the surface area of the electrode in proximity to the tissue, the greater the current density (i.e., the amount of current distributed over an area) of the electrical arc generated by the electrode, and thus the more intense the thermal effect, thereby cutting the tissue. In contrast, the greater the surface area of the electrode in proximity to the tissue, the less the current density of the electrical arc generated by the electrode, thereby coagulating the tissue. Thus, if an electrode having both a broad side and a narrow side is used, e.g., a spatula, the narrow side of the electrode can be placed in proximity to the tissue in order to cut it, whereas the broad side of the electrode can be placed in proximity to the tissue in order to coagulate it. With respect to the characteristics of the electrical energy, as the crest factor (peak voltage divided by root mean squared (RMS)) of the electrical energy increases, the resulting electrical arc generated by the electrode tends to have a tissue coagulation effect. In contrast, as the crest factor of the electrical energy decreases, the resulting electrical arc generated by the electrode tends to have a cutting effect. The crest factor of the electrical energy is typically controlled by controlling the duty cycle of the electrical energy. For example, to accentuate tissue cutting, the electrical energy may be continuously applied to increase its RMS average to decrease the crest factor. In contrast, to accentuate tissue coagulation, the electrical energy may be pulsed (e.g., at a 10 percent duty cycle) to decrease its RMS average to increase the crest factor.

Notably, some electrosurgical generators are capable of being selectively operated in so-called “cutting modes” and “coagulation modes.” This, however, does not mean that the active electrode that is connected to such electrosurgical generators will necessarily have a tissue cutting effect if operated in the cutting mode or similarly will have a tissue coagulation effect if operated in the coagulation mode, since the geometry of the electrode is the most significant factor in dictating whether the tissue is cut or coagulated. Thus, if the narrow part of an electrode is placed in proximity to tissue and electrical energy is delivered to the electrode while in a coagulation mode, the tissue may still be cut.

There are many medical procedures in which tissue is cut or carved away for diagnostic or therapeutic reasons. For example, during hepatic transection, one or more lobes of a liver containing abnormal tissue, such as malignant tissue or fibrous tissue caused by cirrhosis, are cut away. There exists various modalities, including mechanical, ultrasonic, and electrical (which includes RF energy), that can be used to effect resection of tissue. Whichever modality is used, extensive bleeding can occur, which can obstruct the surgeon's view and lead to dangerous blood loss levels, requiring transfusion of blood, which increases the complexity, time, and expense of the resection procedure. To prevent extensive bleeding, hemostatic mechanisms, such as blood inflow occlusion, coagulants, and energy coagulation (e.g., electrosurgical coagulation or argon-beam coagulation), can be used.

In the case where an electrosurgical coagulation means is used, the bleeding can be treated or avoided by coagulating the tissue in the treatment areas with an electro-coagulator that applies a low level current to denature cells to a sufficient depth without breaking intercellular bonds, i.e., without cutting the tissue. Because of their natural coagulation capability, ease of use, and ubiquity, electrosurgical modalities are often used to resect tissue.

During a typical electrosurgical resection procedure, electrical energy can be conveyed from an electrode along a resection line in the tissue. The electrode may be operated in a manner that incises the tissue along the resection line, or coagulates the tissue along the resection line, which can then be subsequently dissected using the same coagulation electrode or a separate tissue dissector to gradually separate the tissue. In the case where an organ is resected, application of RF energy divides the parenchyma, thereby skeletonizing the organ, i.e., leaving vascular tissue that is typically more difficult to cut or dissect relative to the parenchyma.

When a blood vessel is encountered, RF energy can be applied to shrink the collagen in the blood vessel, thereby closing the blood lumen and achieving hemostasis. The blood vessel can then be mechanically transected using a scalpel or scissors without fear of blood loss. In general, for smaller blood vessels less than 3 mm in diameter, hemostasis may be achieved within 10 seconds, whereas for larger blood vessels up to 5 mm in diameter, the time required for hemostasis increases to 15-20 seconds. During or after resection of the tissue, RF energy can be applied to any “bleeders” (i.e., vessels from which blood flows or oozes) to provide complete hemostasis for the resected organ.

When electrosurgically resecting tissue, care must be taken to prevent the heat generated by the electrode from charring the tissue, which generates an undesirable odor, results in tissue becoming stuck on the electrosurgical probe, and most importantly, increases tissue resistance, thereby reducing the efficiency of the procedure. Adding an electrically conductive fluid, such as saline, to the electrosurgery site cools the electrode and keeps the tissue temperature below the water boiling point (100° C.), thereby avoiding smoke and reducing the amount of charring.

Although the application of electrically conductive fluid to the electrosurgery site generally increases the efficiency of the RF energy application, energy applied to an electrode may rapidly diffuse into fluid that has accumulated and into tissue that has already been removed. As a result, if the fluid and removed tissue is not effectively aspirated from the tissue site, the electrosurgery may either be inadequately carried out, or a greater than necessary amount of energy must be applied to the electrode to perform the surgery. Increasing the energy used during electrosurgery increases the chance that adjacent healthy tissues may be damaged. At the same time that fluid accumulation is avoided, care must be taken to ensure that fluid is continuously flowed to the tissue site to ensure that tissue charring does not take place. For example, if flow of the fluid is momentarily stopped, e.g., if the port on the fluid delivery device becomes clogged or otherwise occluded, RF energy may continue to be conveyed from the electrode, thereby resulting in a condition where tissue charring may occur.

There, thus, remains a need to provide a more efficient means for electrosurgically resecting vascularized tissue, while preventing tissue charring and maintaining hemostasis at the treatment site.

### SUMMARY

Electrosurgical devices and systems having one or more porous electrodes are provided. An electrosurgical apparatus is provided having a handle, a shaft, and at least one porous electrode. The shaft is coupled to the handle and the at least one porous electrode is disposed on a distal tip of the shaft. The porous electrode is configured to conduct electrosurgical energy provided to the distal tip to patient tissue disposed adjacent to the electrode. Furthermore, the porous electrode is configured to enable a fluid to pass through the porous structure of the electrode and to be provided to the patient tissue adjacent to the electrode. The electrosurgical apparatus is configured to provide at least a first or second fluid to the distal tip, such as saline or helium, to achieve differing effects.

In one aspect of the present disclosure, the electrosurgical apparatus is configured as a monopolar device having a single electrode.

In another aspect of the present disclosure, the electrosurgical apparatus is configured as a bipolar device having a first electrode and a second electrode.

In another aspect of the present disclosure, the electrosurgical apparatus includes a switching means configured to enable a user to select which of the at least first or second fluids is provided to the distal tip.

In another aspect of the present disclosure, an electrosurgical generator is provided including a switching means configured to selectively provide one of the at least first or second fluids to the electrosurgical apparatus in response to at least one control signal.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is an illustration of an exemplary electrosurgical system including a monopolar electrosurgical apparatus in accordance with an embodiment of the present disclosure;

FIG. 2 is a cross-section view of the monopolar electrosurgical apparatus of FIG. 1 in accordance with an embodiment of the present disclosure;

FIG. 3 is a cross-section view of a bipolar electrosurgical apparatus for use with the electrosurgical system of FIG. 1 in accordance with an embodiment of the present disclosure;

FIG. 4A is an illustration of another exemplary electrosurgical system including a monopolar electrosurgical apparatus in accordance with an embodiment of the present disclosure;

FIG. 4B is a cross-section view of the monopolar electrosurgical apparatus of FIG. 4A in accordance with an embodiment of the present disclosure;

FIG. 4C is an illustration of an electrosurgical generator of the electrosurgical system of FIG. 4A in accordance with an embodiment of the present disclosure;

FIG. 4D is a cross-section view of another bipolar electrosurgical apparatus in accordance with an embodiment of the present disclosure;

FIG. 4E is an illustration of an electrosurgical generator of for use with the bipolar electrosurgical apparatus of FIG. 4D in accordance with an embodiment of the present disclosure;

FIG. 5 is a cross-section view of a monopolar electrosurgical apparatus in accordance with another embodiment of the present disclosure; and

FIG. 6 is a cross-section view of a bipolar electrosurgical apparatus in accordance with another embodiment of the present disclosure.

It should be understood that the drawings are for purposes of illustrating the concepts of the disclosure and are not necessarily the only possible configuration for illustrating the disclosure.

### DETAILED DESCRIPTION

Preferred embodiments of the present disclosure will be described hereinbelow with reference to the accompanying drawings. In the following description, well-known functions or constructions are not described in detail to avoid obscuring the present disclosure in unnecessary detail. In the drawings and in the description which follow, the term “proximal”, as is traditional, will refer to the end of the device, e.g., probe, instrument, apparatus, applicator, handpiece, forceps, etc., which is closer to the user, while the term “distal” will refer to the end which is further from the user. Herein, the phrase “coupled” is defined to mean directly connected to or indirectly connected with through one or more intermediate components. Such intermediate components may include both hardware and software based components.

Devices and systems including one or more porous electrodes are provided. In one embodiment, an electrosurgical apparatus is provided having a shaft, a handle, and at least one porous electrode. The shaft is coupled to the handle and the at least one porous electrode is coupled to a distal tip of the shaft. The at least one porous electrode is configured to conduct electrosurgical energy provided to the distal tip and enable fluid provided to the distal tip to pass through the porous structure of the at least one electrode, such that the electrosurgical energy and the fluid may be simultaneously applied to patient tissue adjacent to the at least one porous electrode. In one embodiment, the electrosurgical apparatus includes a switching means configured to enable a user to select which of at least a first or a second fluid is provided to the distal tip. In another embodiment, an electrosurgical generator is provided including a switching means configured to selectively provide one of the at least first or second fluids to the electrosurgical apparatus responsive to at least one control signal.

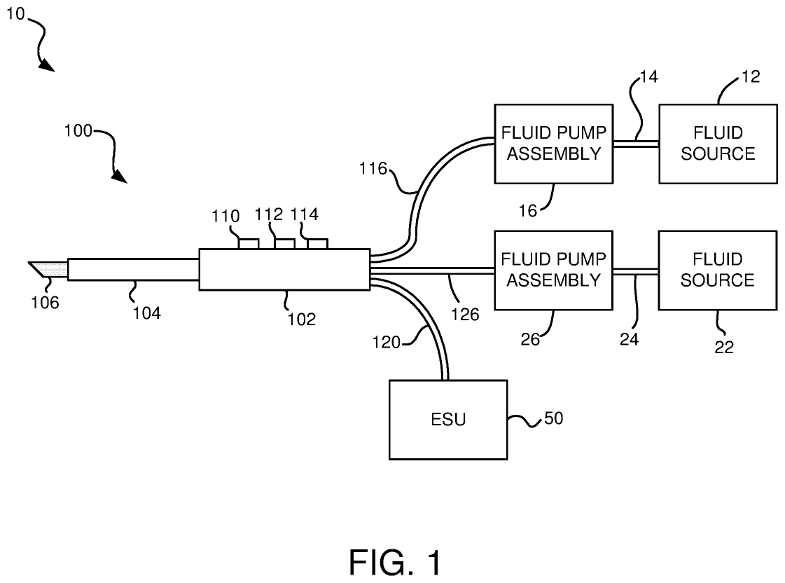

Referring to FIG. 1, an electrosurgical system 10 is shown in accordance with the present disclosure. The system 10 of FIG. 1, includes an electrosurgical apparatus 100 configured for performing various electrosurgical procedures on patient tissue (e.g., cutting, coagulation, ablation, etc.), a fluid pump assembly 16 configured for providing a first fluid (e.g., an electrically conducting fluid, such as saline) received from a first fluid source 12 to apparatus 100, a fluid pump assembly 26 configured for providing a second fluid (e.g., an inert gas, such as helium) received from a second fluid source 22 to apparatus 100, and an electrosurgical generator 50 configured for providing suitable energy to apparatus 100.

Apparatus 100 is coupled to fluid pump assembly 16 via fluid tube 116, fluid pump assembly 26 via fluid tube 126, and electrosurgical generator 50 via cable 120. Assembly 16 is coupled to a first fluid source 12, e.g., saline, via a fluid tube 14 and assembly 26 is coupled to a second fluid source 22, e.g., helium, via a fluid tube 24. Assemblies 14, 16 each include respective fluid gathering and delivery mechanisms (e.g., fluid pumps or other suitable mechanisms) for gather fluid from respective sources 14, 22, and delivering the respective fluid to electrosurgical apparatus 100 via tubes 116, 126.

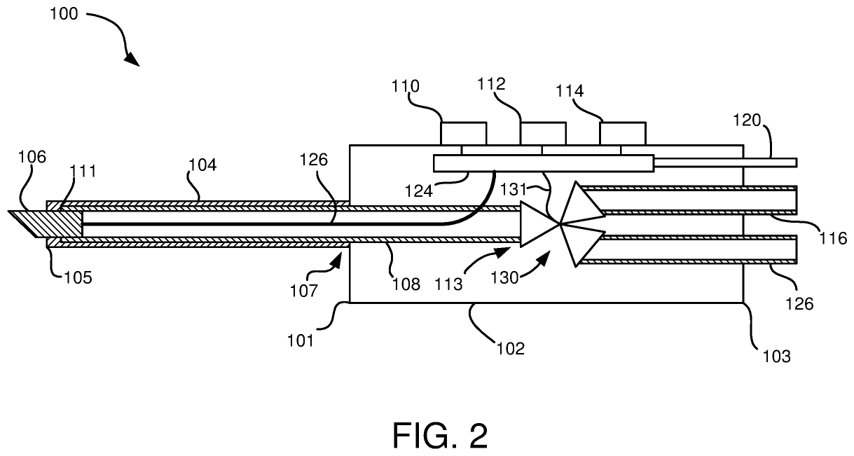

Apparatus 100 includes a handle housing 102, shaft 104, and electrode 106. Referring to FIG. 2, a cross-section view of apparatus 100 is shown in accordance with the present disclosure. Handle 102 includes distal end 101 and proximal end 103. A control circuit 124 is disposed within handle 102 and is coupled to input receiving members 110, 112, 114, where members 110, 112, 114 are each disposed through an outer wall of handle 102. In one embodiment, members 110, 112 are configured as buttons and member 114 is configured as a slider, however, in other embodiments, members 110, 112, 114 may be configured as any type of user selectable control or input receiving means. Cable 120 and tubes 116, 126 are each disposed through proximal end 103 of handle 102. Cable 120 is coupled to circuit 124 and each of tubes 116, 126 are coupled to a 3-way connector switch 130 (e.g., in one embodiment, a 3-way fluid valve), which will be described in greater detail below.

Shaft 104 includes a distal end 105 and a proximal end 107. Proximal end 107 of shaft 104 is coupled to distal end 101 of handle 102, such that, shaft 104 extends distally from handle 102. Shaft 104 is configured as a tube having a hollow interior and made of an insulative material. A flow tube 108, configured for carrying a fluid to the distal end of shaft 104 is disposed though the interior of shaft 104. A proximal end 113 of flow tube 108 is coupled to connector switch 130 and a distal end 111 of flow tube 108 is coupled a proximal portion of electrode 106. In one embodiment, electrode 106 is configured as a planar blade having a tapered distal point and a beveled edge, such that electrode 106 is suitable for both electrosurgical cutting (when energized) and mechanical cutting (when de-energized).

In one embodiment, flow tube 108 is made of an insulative material and a conductive wire 120 is coupled to circuit 124 and disposed through a wall of flow tube 108 and into the interior of flow tube 108. Wire 126 extends within the interior of flow tube 108 and is coupled to a proximal portion of electrode 106. Circuit 124 is configured to receive electrosurgical energy (e.g., a radio frequency waveform) from electrosurgical generator 50 via cable 120. When button 110 is pressed by a user, circuit 124 enables the electrosurgical energy received via cable 120 to be applied to wire 126 and thus to electrode 106. It is to be appreciated that electrosurgical generator 50 may be configured with various waveforms configured to provide differing tissue effects when applied to electrode 106. In another embodiment, flow tube 108 may be made of a conductive material and wire 126 may be coupled to a proximal portion of tube 108. In this embodiment, flow tube 108 conducts electrosurgical energy to electrode 106 when button 110 is pressed.

In either case, circuit 124 is coupled to connector 130, via line 131, and configured to change the state of connector 130 when a user presses button 112. In a first state, connector 130 is configured to enable the first fluid from tube 116 to flow through connector 130 and into tube 108 while blocking the second fluid from tube 126 from flowing through connector 130 and into tube 108. In a second state, connector 130 is configured to enable the second fluid from tube 126 to flow through connector 130 and into tube 108 while blocking the first fluid from tube 116 from flowing through connector 130 and into tube 108. In this way, each time button 112 is pressed, the user may select which of the first fluid or the second fluid is provided to electrode 106. It is to be appreciated that, in some embodiments, connector 130 may include a third state, where connector 130 does not enable either the first fluid from tube 116 or the second fluid from tube 118 to pass into tube 108. In the third state, neither the first fluid, nor the second fluid, are provided to electrode 106. The connector 130 may be a mems (microelectromechanical systems) valve, however, other types of valves and/or switching connectors are contemplated to be within the scope of the present disclosure.

In one embodiment, apparatus 100 includes a flow control mechanism (e.g., either integrated in connector 130 or discrete from connector 130) configured to control the flow rate of either the first fluid or the second fluid through tube 108. In this embodiment, when a user engages slider 114 (e.g., slides slider 114 relative to handle 102), circuit 124 sends a signal to the flow control mechanism to selectively change the flow rate of the first fluid or second fluid through tube 108. In one embodiment, the flow control mechanism controls the flow rate of the first fluid or the second fluid through tube 108 by pinching tube 108 to change the diameter of tube 108, thus changing the flow rate. In this embodiment, tube 108 is a flexible tube. In another embodiment, apparatus 100 controls the flow rate of the first fluid or the second fluid by sending a control signal from circuit 124 to assemblies 16, 26 (e.g., via a respective cable coupling circuit 124 to each of assemblies 16, 26) to cause the fluid mechanisms (e.g., variable speed pumps) in assemblies 16, 26 to provide the first fluid or the second fluid at a desired rate.

Electrode 106 is made of a conductive material (e.g., stainless steel) having a porous structure that renders the electrode 106 pervious to the passage of fluid (e.g., the first fluid or the second fluid provided via tube 108), thereby facilitating the uniform distribution of an electrically conductive fluid into the tissue during, for example, the ablation process. The porous structure allows fluid to pass through the electrode 106. In addition to providing a more uniform distribution of fluid, the porous structure of electrode 106 is configured such that tissue is less apt to stick to the surfaces of the electrode 106 while electrode 106 is in use and a fluid, such as saline (e.g., the first fluid received via tube 116 and tube 108), is provided through the pores of electrode 106.

To this end, the porous structure of electrode 106 comprises a plurality of pores that are in fluid communication with the interior of flow tube 108. In one embodiment, the pores of the porous structure are interconnected in a random, tortuous, interstitial arrangement to maximize the porosity of the electrodes 106. The porous structure may be microporous, in which case, the effective diameters of the pores are in the 0.05-20 micron range, or the porous structure may be macroporous, in which case, the effective diameters of the pores are in the 20-2000 micron range. In one embodiment, the pore size may be in the 1-50 micron range. The porosity of the porous structure, as defined by the pore volume over the total volume of the structure, may be in the 20-80 percent range. Naturally, the higher the porosity, the more freely the first fluid or the second fluid will flow through the electrodes 106. Thus, the designed porosity of the porous structure will ultimately depend on the desired flow of the first fluid or second fluid through electrode 106.

Thus, it can be appreciated that the pervasiveness of the pores in the porous structure enables the first fluid or the second fluid to freely flow from tube 108, through the thickness of the electrode 106, and out to the tissue adjacent to electrode 106. It is to be appreciated that this free flow of fluid occurs even if several of the pores have been clogged with material, such as tissue. In one embodiment, the porous structure provides for the wicking (i.e., absorption of fluid by capillary action) of fluid into the pores of the porous structure. To promote the wicking of fluid into the porous structure, the porous structure may be hydrophilic.

The porous structure is composed of a metallic material, such as stainless steel, titanium, or nickel-chrome. While electrode 106 is preferably composed of an electrically conductive material, the electrode 106 may alternatively be composed of a non-metallic material, such as porous polymer or ceramic. While the porous polymers and ceramics are generally non-conductive, they may be used to conduct electrical energy to the tissue by virtue of the conductive fluid (e.g., the first or second fluids provided via tubes 116, 126) within the interconnected pores of electrode 106.

In one embodiment, the porous structure is formed using a sintering process, which involves compacting a plurality of particles (preferably, a blend of finely pulverized metal powders mixed with lubricants and/or alloying elements) into the shape of the electrode 106, and then subjecting the blend to high temperatures. When compacting the particles, a controlled amount of the mixed powder is automatically gravity-fed into a precision die and is compacted, usually at room temperature at pressures as low as 10 or as high as 60 or more tons/inch2 (138 to 827 MPa), depending on the desired porosity of the electrode 106. The compacted powder will have the shape of the electrode 106 once it is ejected from the die, and will be sufficiently rigid to permit in-process handling and transport to a sintering furnace. Other specialized compacting and alternative forming methods can be used, such as, but not limited to, powder forging, isostatic pressing, extrusion, injection molding, and spray forming.

During sintering, the unfinished electrode 106 is placed within a controlled-atmosphere furnace, and is heated to below the melting point of the base metal, held at the sintering temperature, and then cooled. The sintering transforms the compacted mechanical bonds between the powder particles to metallurgical bonds. The interstitial spaces between the points of contact will be preserved as pores. The amount and characteristics of the porosity of the structure can be controlled through powder characteristics, powder composition, and the compaction and sintering process.

It is to be appreciated that porous structures can be made by methods other than sintering. For example, pores may be introduced by mechanical perforation, by the introduction of pore producing agents during a matrix forming process, or through various phase separation techniques. Also, the porous structure may be composed of a ceramic porous material with a conductive coating deposited onto the surface, e.g., by using ion beam deposition or sputtering.

The usage of a conductive material including a porous structure for electrode 106 enables a first fluid, such as saline, and a second fluid, such as helium, to be provided to the tissue treated concurrently with the electrosurgical energy applied via be electrode 106 to the patient tissue. Where the first fluid is saline, the effect of providing the first fluid to the tissue adjacent to electrode 106 has many benefits, including, but not limited to, (1) faster, but controlled, dissection, (2) less charring of tissue, (3) electrode 106 remains cleaner during use (i.e., less tissue sticks to electrode 106, resulting in less re-bleeding when electrode 106 is pulled off tissue), (4) less smoke is produced from heated tissue, (5) greater depth of coagulation is realized, and (5) vessel sealing occurs. Where the second fluid is helium, the effect of providing the second fluid to electrode 106 has the benefit of enabling electrode 106 to produce plasma when energized, as will be described below.

In use, shaft 104 of apparatus 100 may be disposed through a cannula or trocar and into a tissue structure of a patient to perform an electrosurgical procedure on patient tissue. When button 110 is pressed, electrosurgical energy received from an energy source, such as, electrosurgical generator 50 is provided via wire 126 to energize electrode 106, such that an electrosurgical effect (e.g., cutting, coagulation, ablation, etc.) is realized when the electrosurgical energy is conducted by electrode 106. Furthermore, via the selection of button 112 and slider 114, a first fluid (e.g., saline) or a second fluid (e.g., helium) received from a respective fluid source is provided to electrode 106 via tube 108. The porous structure enables the first fluid or the second fluid to flow or pass through the porous structure and exit electrode 106 and be applied to patient tissue adjacent to electrode 106.

In one embodiment, the second fluid is an inert gas, such as helium. In this embodiment, when the helium gas is provided to electrode 106 and electrode 106 is energized, plasma is generated and ejected from the pores of electrode 106 as a diffuse plasma cloud and is applied to the patient tissue. It is to be appreciated that in some embodiments, only a selected portion or portions (e.g., a subset of the entire volume) of electrode 106 may be configured with or comprise the porous structure to enable control of the geometry of the diffuse plasma cloud generated by electrode 106 when helium gas is provided. Furthermore, different regions of the porous structure and/or electrode 106 may selectively be configured with various or different levels or amounts of porosity to control how the fluid flows through the electrode 106, where the fluid exits from electrode 106, and how the shape or geometry of a diffuse plasma cloud generated by electrode 106 is determined or selected. Where regions or portions of electrode 106 include zero porosity, no fluid passes through these regions or portions of the electrode.

It is to be appreciated that in the embodiments described above, shaft 104 is configured to be rigid and linear. However, in other embodiments of the present disclosure, shaft 104 may be configured to be flexible to enable shaft 104 to be bent such that distal end 105 of shaft 104 may achieve a variety of different orientations with respect to handle 102. In these embodiments, the flow tube 108 may be configured to be flexible to bend along with the shaft 104; for example, the flow tube 108 may be configured as a spring with a shrink wrap disposed therein to prevent leakage of the fluid. Other flexible materials are contemplated to be within the scope of the present disclosure. In some embodiments, the distal end 105 of shaft 104 (or flow tube 108, where shaft 104 is removed) may be configured to be grasped by forceps of a robotic arm to manipulate the orientation of the distal end 105 of shaft 104 (or fluid tube 108) with respect to handle 102.

Furthermore, it is to be appreciated that, in other embodiments, electrode 106 may be configured in different geometries than shown in FIGS. 1 and 2. For example, electrode 106 may be configured as a needle, ball, or any other geometric shape without deviating from the scope of the present disclosure.

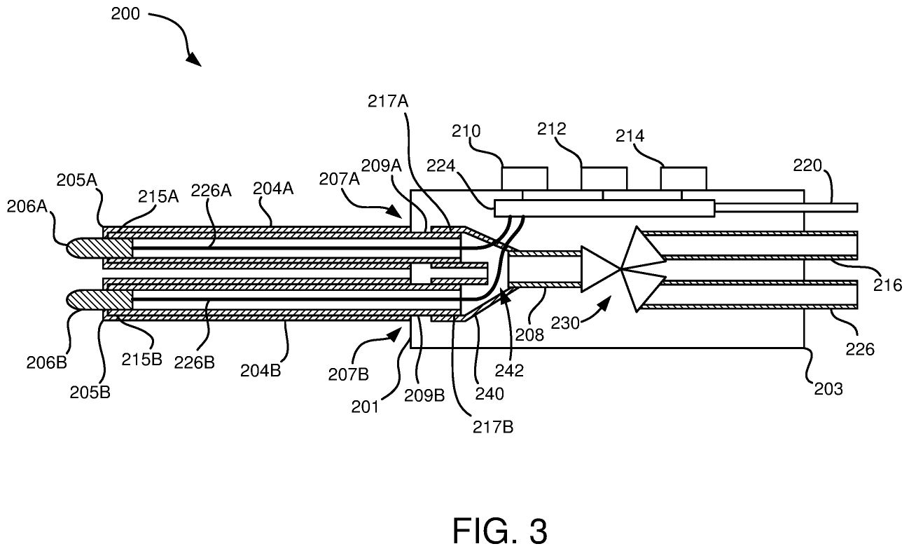

In another embodiment of the present disclosure, apparatus 100 may be modified for bipolar electrosurgical applications. For example, referring to FIG. 3, a cross-section view of an electrosurgical apparatus 200 modified for bipolar use with the system 10 is shown in accordance with the present disclosure. It is to be appreciated that, unless otherwise indicated, components of the apparatus 300 shown in FIG. 3 that are similarly numbered to corresponding components of apparatus 100 shown in FIGS. 1 and 2 (e.g., 108 and 208, 130 and 230, etc.) are configured in the manner and with the features described above and may not be described again below in the interest of brevity.

As shown in FIG. 3, apparatus 200 includes active and return electrodes 206A, 206B, which are each mounted in or coupled to respective distal ends 205A, 205B of respective insulative shafts 204A, 204B. In this embodiment, apparatus 200 further includes y-connector 240, flow or fluid tubes 209A, 209B, and conductive wires 226A, 226B. Flow tubes 209A, 209B are disposed through the distal end 201 and into the interior of shafts 204A, 204B, respectively. The distal end 215A of flow tube 209A is coupled to a proximal portion of electrode 206A and the distal end 215B of flow tube 209B is coupled to a proximal portion of electrode 206B. In one embodiment, electrodes 206A, 206B are configured with blunted distal ends. The proximal ends 217A, 217B of each of flow tubs 209A, 209B are coupled to the distal end of y-connector 240 and disposed through respective fluid channels of y-connector 240. The distal end of flow tube 208 is coupled to the proximal end of y-connector 240 and disposed in an interior channel 242 of y-connector 240. When the first fluid or the second fluid is provided from tube 216 or tube 226 via connector 230 to channel 242 of y-connector 240, the provided fluid is split within connector 240 and provided to the interior of each of flow tubes 209A, 209B and via tubes 209A, 209B to electrodes 206A, 206B.

In one embodiment, tubes 209A, 209B are made of an insulative material and wires 226A, 226B are coupled to circuit 224 and each extend through a wall of connector 240 and into channel 240. From channel 240, wire 226A extends into the interior of tube 209A and is coupled to electrode 206A and wire 226B extends into the interior of tube 209B and is coupled to electrode 206B. In another embodiment, tubes 209A, 209B are made of a conductive material for providing electrosurgical energy to electrodes 206A, 206B and wire 226A is coupled to a proximal portion of tube 209A for providing electrosurgical energy thereto and wire 226B is coupled to a proximal portion of tube 209B for providing electrosurgical energy thereto.

In either case, when button 210 is pressed, electrosurgical energy provided via cable 220 is applied to wire 226A and via wire 226A to electrode 206A. As described above, apparatus 200 is configured for bipolar applications. When each of electrodes 206A, 206B is in contact with tissue, energy is provided to electrode 206A, passed through the target tissue and is returned via electrode 206B and wire 226B to cable 220 to be provided to the electrosurgical generator 50. It is to be appreciated that each of electrodes 206A, 206B may be configured to alternate between acting as the active electrode or the return electrode for bipolar applications.

It is to be appreciated that each of electrodes 206A, 206B are configured with material having a porous structure (e.g., in the manner described above with respect to electrode 106 in FIGS. 1 and 2), such that when the first fluid (e.g., saline) or the second fluid (e.g., helium) is provided through each of tubes 209A, 209B, the fluid passes through each of electrodes 206A, 206B and is applied to patient tissue. As described above, in one embodiment, the second fluid is an inert gas, such as helium. In this embodiment, when the second fluid is provided to each of electrodes 206A, 206B, and energy is applied across electrodes 206A, 206B, plasma is generated and ejected through the porous structure of electrodes 206A, 206B in the form of a diffuse plasma cloud and is applied to patient tissue adjacent to electrodes 206A, 206B. It is to be appreciated that the path the generated plasma takes when ejected depends on the spacing of electrodes 206A, 206B with respect to each other, the patient tissue adjacent to electrodes 206A, 206B and the resistance of the proximate patent tissue. The plasma generated by apparatus 200 will take the path of least resistance. As such, if the path of least resistance for the plasma is the direct path between electrodes 206A, 206B, the generated plasma will arc between electrodes 206A, 206B. In certain embodiments, the plasma arc formed between electrodes 206A, 206B may be employed to cut tissue akin to using a wire to cut through the tissue or to remove a portion of tissue, e.g., to slice off a thin layer of tissue at the surgical site. Alternatively, if there is patient tissue between electrodes 206A, 206B, the generated plasma will flow through a region of the patient tissue adjacent to electrodes 206A, 206B.

It is to be appreciated that in the embodiments described above, shafts 204A, 204B are configured to be rigid and linear. However, in other embodiments of the present disclosure, shafts 204A, 204B may be configured to be flexible to enable shafts 204A, 204B to be bent such that distal ends of shafts 204A, 204B may achieve a variety of different orientations with respect to handle 202. In these embodiments, the flow tubes 209A, 209B may be configured to be flexible to bend along with the shafts 204A, 204B; for example, the flow tubes 209A, 209B may be configured as a spring with a shrink wrap disposed therein to prevent leakage of the fluid. Other flexible materials are contemplated to be within the scope of the present disclosure. In some embodiments, the distal ends of shafts 204A, 204B (or of fluid tubes 209A, 209B, where shafts 204A, 204B are removed) may be configured to be grasped by forceps of a robotic arm to manipulate the orientation of the distal ends of shafts 204A, 204B (or fluid tubes 209A, 209B) with respect to handle 202. In some embodiments, shaft 204A, 204B are coupled together, such that shafts 204A, 204B are manipulated in unison. In other embodiments, shafts 204A, 204B not coupled together, such that, each of shafts 204A, 204B are free to be manipulated independently of each other using forceps.

In another embodiment of the present disclosure, connectors 130 may be removed from apparatus 100 and connector 230 may be removed form apparatus 200 and electrosurgical generator 50 may be configured to selectively provide the first fluid or the second fluid to apparatus each of apparatuses 100 and 200 in addition to electrosurgical energy via a single cable.

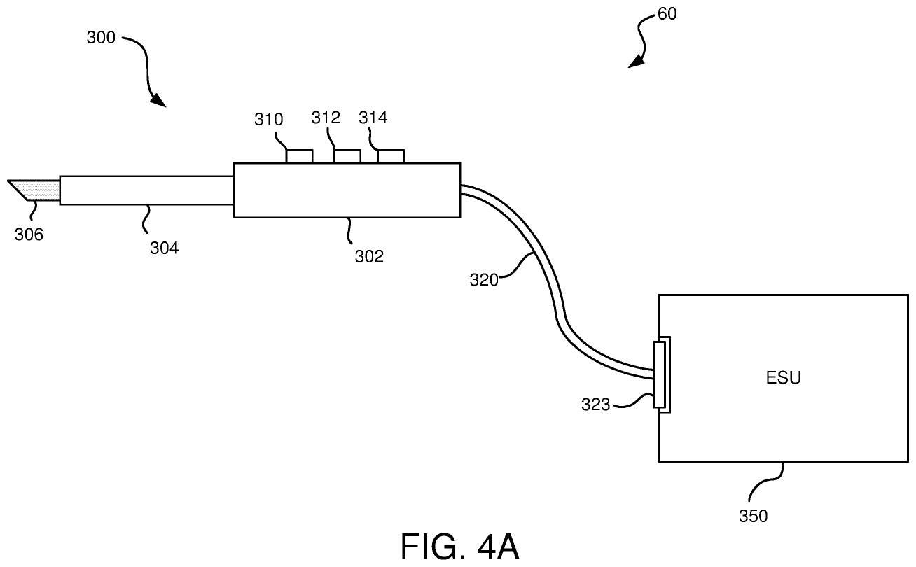

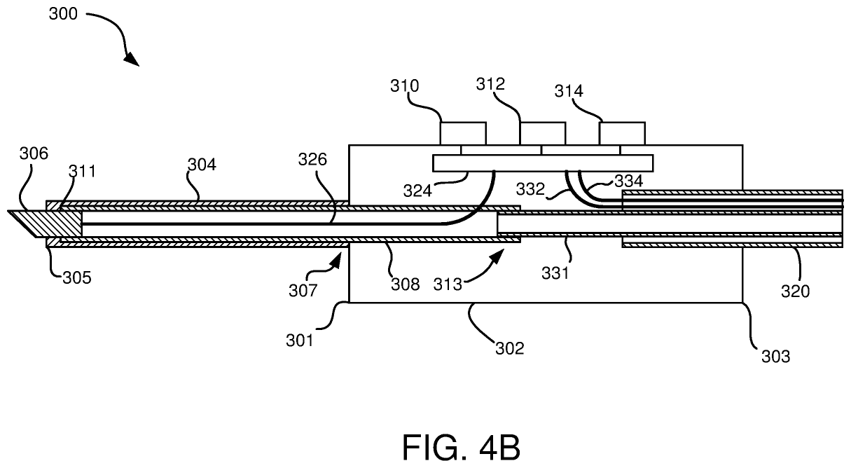

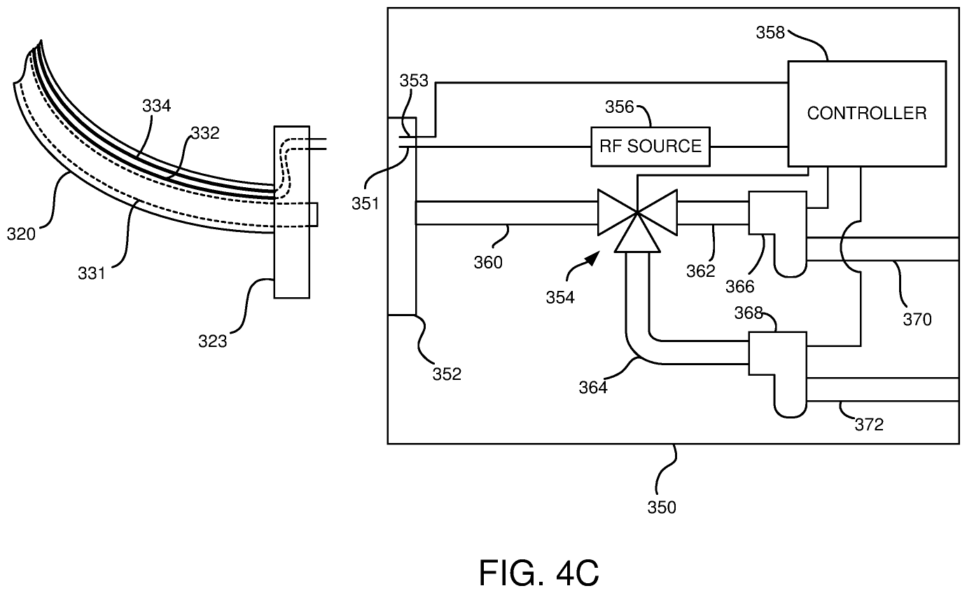

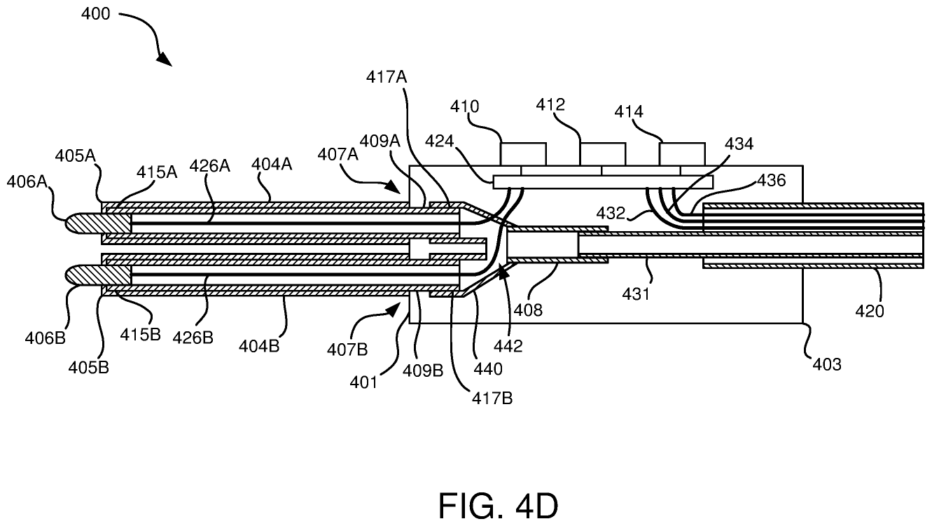

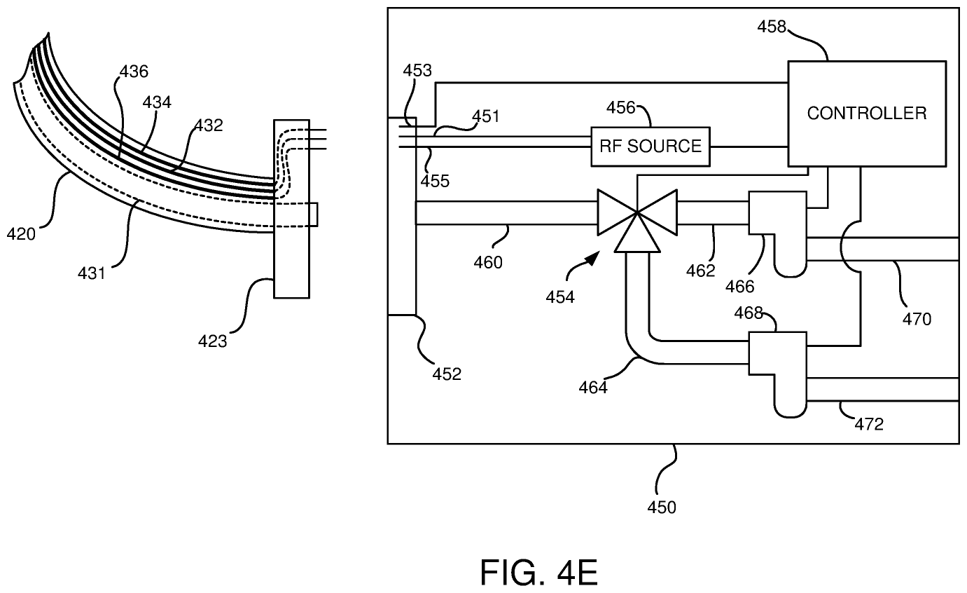

For example, referring to FIGS. 4A, 4B, 4C a system 60 is shown, where a monopolar apparatus 300 including a porous electrode 306 is coupled to electrosurgical generator 350 via a cable 320 and a connector 323. It is to be appreciated that, unless otherwise indicated, components of the apparatus 300 and electrosurgical generator 350 shown in FIGS. 4A, 4B, 4C that are similarly numbered to corresponding components of apparatus 100 shown in FIGS. 1 and 2 (e.g., 208 and 308, 230 and 330, etc.) are configured in the manner and with the features described above and may not be described again below in the interest of brevity.

As shown in FIGS. 4A, 4B, cable 320 includes conducting wires 332, 334 and a flexible flow tube 331. It is to be appreciated that although only two conducting wires 332, 334 are shown in cable 320, cable 320 may include any number of conducting wires without deviating from the scope of the present disclosure. Wires 332, 334 are each coupled to circuit 324 in handle 302 of apparatus 320 and tube 331 is coupled to the proximal end 313 of tube 308. Referring to FIG. 4C, in this embodiment, electrosurgical generator 350 includes a receptacle 352 configured to receive connector 323. When receptacle 352 receives connector 323, wire 332 is electrically coupled to a conductive pin 351 of receptacle 352, wire 334 is electrically coupled to a conductive pin 353, and flow tube 331 is coupled to a flow tube 360, which extends from receptacle 352 into the interior of generator 350.

As shown in FIG. 4C, generator 350 includes fluid tubes 360, 362, 364, 370, 372, fluid pumps 366, 368, connector 354, RF energy source (e.g., including one or more transformers for generating radio frequency waveforms) 356, and one or more controllers or processors 358. Controller 358 is configured to control the components of generator 350 and is coupled to pin 353, connector 354, RF source 356, pump 366, and pump 368. RF energy source 356 is further coupled to pin 351. Tube 360 is coupled to receptacle 352 and connector 354, where connector 354 is further coupled to tubes 362, 364. Tube 362 is further coupled to pump 366 and tube 364 is further coupled to pump 368. Pump 366 is further coupled to tube 370 and pump 368 is further coupled to tube 372. Tube 370 is configured to receive the first fluid (e.g., saline) from assembly 16 or directly from fluid source 12. Tube 372 is configured to receive the second fluid (e.g., helium) from assembly 26 or directly from fluid source 22.

Controller 358 is configured to control energy source 356, connector 354, and pumps 366, 368 responsive to one or more control signals received from pin 353 when a user presses buttons 310, 312, or operates slider 314. Controller may control the components of generator 350 based on instructions stored on controller 358 or one or more memory devices coupled to controller 358. Pump 366 is configured to gather the first fluid from tube 370 and provide the first fluid to tube 362 at a flow rate selected by controller 358. Pump 368 is configured to gather the second fluid from tube 372 and provide the second fluid to tube 364 at a flow rate selected by controller 358. Connector 354 is configured to enter a first state or a second state responsive to at least one signal received by controller 358. In the first state, connector 354 enables or allows the first fluid to flow from tube 362, through connector switch 354, and into tube 360 at the flow rate selected by controller 358 and connector 354 blocks the second fluid from flowing from tube 364 to tube 360. In the second state, connector 354 enables or allows the second fluid to flow from tube 364, through connector switch 354, and into tube 360 at the flow rate selected by controller 358 and connector 354 blocks the first fluid from flowing from tube 362 to tube 360. Based on the state of connector 354, the first fluid or the second fluid is provided from connector 354 to tube 360, to tube 331, to tube 308, and to porous electrode 306. It is to be appreciated that connector 354 may be 3-way valve and/or a mems (microelectromechanical systems) valve, however, other types of valves and/or switching connectors are contemplated to be within the scope of the present disclosure.

When button 310 of apparatus 300 is pressed, at least one first control signal is generated by circuit 324 and provided via wire 334 and pin 353 to controller 358. Responsive to the first control signal, controller 358 is configured to cause RF energy source 356 to provide electrosurgical energy via pin 351 and wire 332 to circuit 324, where the electrosurgical energy is further applied to electrode 306 via wire 326 (or, where tube 308 is conducting, via tube 308). When button 312 of apparatus 300 is pressed, at least one second control signal is generated by circuit 324 and provided via wire 324 and pin 353 to controller 358. Responsive to the second control signal, controller 358 is configured to cause connector 354 to switch between the first state or the second state, such that either the first fluid is provided to tube 360 or the second fluid is provided to tube 360 through connector 354. It is to be appreciated that, when controller 358 switches to the first state or the second state, the appropriate pump 366, 368 is activated to draw or pump the first fluid or the second fluid through connector 354 and tube 360. When button 314 of apparatus 300 is pressed, a third control signal is generated by circuit 324 and provided via wire 334 and pin 353 to controller 358. Responsive to the third control signal, controller 358 is configured to selectively change the flow rate that pump 366 provides the first fluid through tube 362 or the flow rate that pump 368 provides the second fluid through tube 364.

It is to be appreciated that, generator 350 may be configured for use with a bipolar electrosurgical apparatus, such as apparatus 200 described above. For example, referring to FIGS. 4D and 4E, in FIG. 4E a bipolar electrosurgical apparatus 400 having porous electrodes 406A, 406B is shown and in FIG. 4E an electrosurgical generator 450 for selectively providing the first fluid, the second fluid, and electrosurgical energy to apparatus 400 is shown in accordance with an embodiment of the present disclosure. It is to be appreciated that, unless otherwise indicated, components of the apparatus 400 and electrosurgical generator 450 shown in FIGS. 4A, 4B, 4C that are similarly numbered to corresponding components of apparatuses 100, 200, 300 shown in FIGS. 1, 2, 3, and 4A-4C are configured in the manner and with the features described above and may not be described again below in the interest of brevity.

As shown in FIGS. 4D and 4E, bipolar apparatus 400 includes an additional wire 436, which is coupled to circuit 424, and receptacle 452 includes an additional pin 455, which is coupled to RF energy source 456. Return electrode 406B is coupled via wire 426B and wire 436 to pin 455, such that a closed circuit is formed between energy source 456 and electrodes 406A, 406B for bipolar applications.

It is to be appreciated that in the embodiments of electrosurgical generators 350, 450 shown above in FIGS. 4C and 4E, pumps 366, 368 and/or 466, 468 may be disposed externally to generators 350, 450, e.g., pumps 366, 466 may be disposed in assembly 16 and pumps 368, 468 may be disposed in assembly 26.

It is to be appreciated that the systems described above, including any of electrosurgical apparatuses 100, 200, 300, 400 and generators 50, 250, 450, may be used for various electrosurgical procedures where it is advantageous to selectively provide a first fluid or gas (e.g., saline), a second fluid or gas (e.g., helium), and/or electrosurgical energy to one or more electrodes (e.g., having porous structures, as described above) at different points throughout the procedure. In one embodiment, a method for using apparatuses 100, 200, 300, 400 and/or generators 50, 350, 450 includes:

(1) Applying a first fluid (e.g., saline) and electrosurgical energy to patient tissue via one or more electrodes to perform ablation at the surgical site. For example, a user may engage one or more controls (e.g., button 110, 112, 210, 212, 310, 312, 410, 412) of an electrosurgical apparatus (e.g., 100, 200, 300, 400) to cause the first fluid to be provided via a flow tube (e.g., 108, 209, 308, 409) to one or more electrodes (e.g., 106, 206, 306, 406) and for electrosurgical energy to be applied to the one or more electrodes. The first fluid first fluid passes through a porous structure (as described above) in each of the one or more electrodes and is applied to patient tissue.

(2) Ceasing the application of the first fluid and electrosurgical energy to the patient tissue. For example, a user may engage a user control (e.g., 112, 212, 312, 412) of the electrosurgical apparatus to cause the flow of the first fluid through the flow tube to cease.

(3) Applying a second fluid or gas (e.g., helium) along the flow tube (e.g., 108, 209, 308, 409) to the patient tissue to clear the flow tube (e.g., 108, 209, 308, 409) and patient tissue of the first fluid and other materials/substances. For example, a user may engage a control (e.g., button 114, 214, 314, 414) of the electrosurgical apparatus (100, 200, 300, 400) to cause the second fluid to be provided via the flow tube (e.g., 108, 209, 308, 409) to one or more electrodes (106, 206, 306, 406). The second fluid passes through the porous structure in each of the one or more electrodes and is applied to patient tissue.

(4) Applying the second fluid or gas and electrosurgical energy to the one or more electrodes to generate a plasma cloud to be applied to the ablated patient tissue. For example, a user may engage one or more controls (e.g., buttons 110, 114, 210, 214, 310, 314, 410, 414) of the electrosurgical apparatus (100, 200, 300, 400) to cause electrosurgical energy to be applied to the one or more electrodes (e.g., 106, 206, 306, 406) and the second fluid to be provided via the flow tube (e.g., 108, 209, 308, 409), such that the electrosurgical energy and the second fluid is applied to the patient tissue.

It is to be appreciated that the electrosurgical apparatuses 100, 200, 300, 400 of the present disclosure enable a user to selectively apply a first fluid or a second fluid and/or electrosurgical energy to patient tissue by engaging one or more user controls (e.g., 110, 112, 114, 210, 212, 214, 310, 312, 314, 410, 412, 414). In this way, any of the steps described in the method above may be removed, reordered, and/or performed in isolation during different procedures. For example, a user may use apparatuses 100, 200, 300, 400 to apply electrosurgical energy and the first fluid to patient tissue during a procedure without applying the second fluid at any point during the procedure. Alternatively, a user may use apparatuses 100, 200, 300, 400 to apply electrosurgical energy and the second fluid to patient tissue during a different procedure without applying the first fluid at any point during the procedure.

In another embodiment of the present disclosure, connector 130 may be removed from apparatus 100 and connector 230 may be removed form apparatus 200 and only inert gas may be provided to the flow tubes of each of apparatuses 100 and 200.

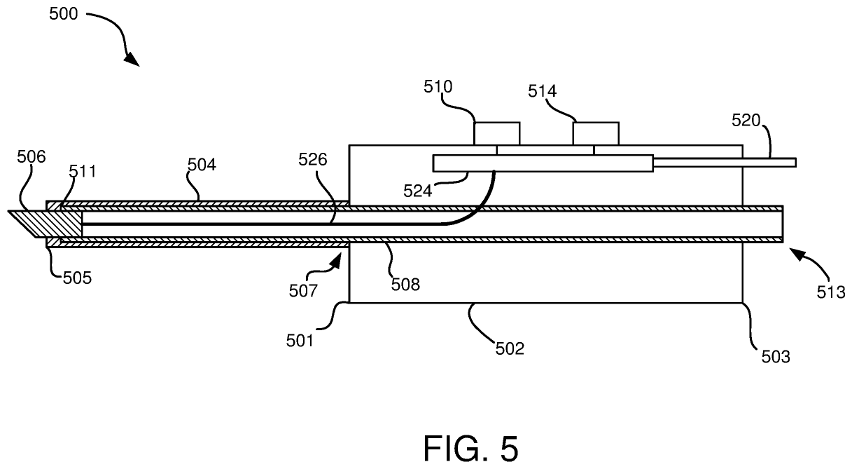

For example, referring to FIG. 5, a monopolar apparatus 500 including a porous electrode 506 is shown in accordance with an embodiment of the present disclosure. It is to be appreciated that, unless otherwise indicated, components of the apparatus 500 shown in FIG. 5 that are similarly numbered to corresponding components of apparatus 100 shown in FIGS. 1 and 2 (e.g., 108 and 508, 106 and 506, etc.) are configured in the manner and with the features described above and may not be described again below in the interest of brevity.

As shown in FIG. 5, flow or fluid tube 508 extends through housing 502 and into shaft 504. The proximal end 513 of tube 508 is configured to receive inert gas (e.g. helium) and provide the helium to porous electrode 506. When button 510 is pressed, energy received from an electrosurgical generator (such as any of the generators described above) via wire 520 is provided via wire 526 to electrode 506, such that when inert gas is provided to electrode 506, a diffuse plasma cloud is emitted via the pours of electrode 506 (e.g., as described above in other embodiments). A slider 514 may be used by a user to select the fluid flow rate of the inert gas provided to electrode 506.

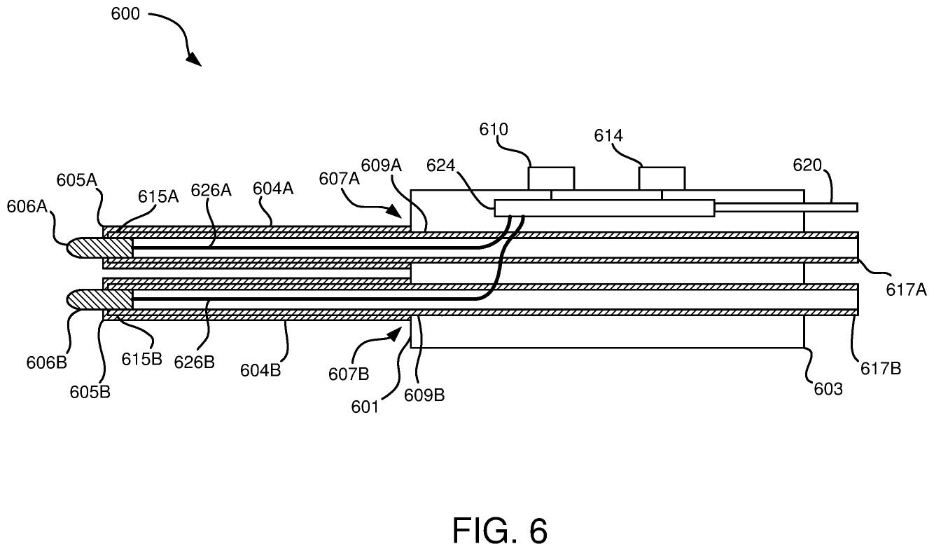

Referring to FIG. 6, a bipolar apparatus 600 including porous electrodes 606A, 606B is shown in accordance with an embodiment of the present disclosure. It is to be appreciated that, unless otherwise indicated, components of the apparatus 600 shown in FIG. 6 that are similarly numbered to corresponding components of apparatus 200 shown in FIG. 3 (e.g., 209 and 609, 206 and 606, etc.) are configured in the manner and with the features described above and may not be described again below in the interest of brevity.

As shown in FIG. 6, flow or fluid tubes 609A, 609B extend through housing 602 and into respective shafts 604A, 604B. The proximal ends 617A, 617B of tubes 609A, 609B are configured to receive inert gas (e.g. helium) from a fluid source and provide the helium to porous electrodes 606A, 606B. When button 610 is pressed, energy received from an electrosurgical generator (such as any of the generators described above) via wire 620 is provided via wires 626A, 626B to electrodes 606A, 606B, such that when inert gas is provided to electrodes 606A, 606B, diffuse plasma clouds are emitted via the pours of electrodes 606A, 606B (e.g., as described above in other embodiments). A slider 614 may be used by a user to select the fluid flow rate of the inert gas provided to electrodes 606A, 606B.

In one aspect of the present disclosure, an electrosurgical apparatus is provided comprising: a handle including an interior, a proximal end, and a distal end; a fluid tube including a proximal end and a distal end, the proximal end of the fluid tube disposed through the distal end of the handle into the interior of the handle; at least one porous electrode coupled to the distal end of the fluid tube; and a connector switch disposed in the interior of the handle and coupled to the proximal end of the fluid tube, the connector switch configured to receive at least one first fluid from a first fluid source and at least one second fluid from a second fluid source and, responsive to a user input, provide one of the at least one first fluid or the at least one second fluid to the fluid tube, the fluid tube configured to provide the fluid to the at least one porous electrode, wherein the at least one porous electrode includes a porous structure configured to allow fluid provided via the fluid tube to flow through the porous structure and exit the at least one porous electrode, wherein the at least one porous electrode is configured to receive and conduct electrosurgical energy from an energy source.

In one aspect, the electrosurgical apparatus is provided, wherein the at least one first fluid is an electrically conducting fluid.

In one aspect, the electrosurgical apparatus is provided, wherein the electrically conducting fluid is saline.

In one aspect, the electrosurgical apparatus is provided, wherein the at least one second fluid is an inert gas.

In one aspect, the electrosurgical apparatus is provided, wherein when the at least one porous electrode is energized and the inert gas is provided to the at least one porous electrode, plasma is generated and ejected from the at least one porous electrode.

In one aspect, the electrosurgical apparatus is provided, wherein the plasma is ejected as a diffuse plasma cloud.

In one aspect, the electrosurgical apparatus is provided, wherein the porous structure of the at least one porous electrode comprises a subset of the entire volume of the at least one porous electrode to control the geometry of the generated diffuse plasma cloud.

In one aspect, the electrosurgical apparatus is provided, wherein different regions of the porous structure are selectively configured with different levels of porosity to control the geometry of the generated diffuse plasma cloud.

In one aspect, the electrosurgical apparatus is provided, wherein the inert gas is helium.

In one aspect, the electrosurgical apparatus is provided, wherein the connector switch is a 3-way fluid valve.

In one aspect, the electrosurgical apparatus is provided, wherein the connector switch is a microelectromechanical system valve.

In one aspect, the electrosurgical apparatus is provided, wherein the fluid tube includes an outer wall and an interior and the electrosurgical apparatus further comprises a conductor disposed through the outer wall and into the interior of the fluid tube and coupled to the at least one porous electrode, the conductor configured to receive electrosurgical energy from the energy source and provide the electrosurgical energy to the at least one porous electrode.

In one aspect, the electrosurgical apparatus is provided, wherein the fluid tube is made of a conductive material and configured to receive electrosurgical energy from the energy source and provide the electrosurgical energy to the at least one porous electrode.

In one aspect, the electrosurgical apparatus further comprises a circuit configured to change a state of the connector switch, wherein in a first state the connector switch is configured to enable the at least one first fluid to be provided to the at least one porous electrode via the fluid tube and to block the at least one second fluid from flowing through the connector switch and in a second state the connector switch is configured to enable the at least one second fluid to be provided to the at least one porous electrode via the fluid tube and to block the at least one first fluid from flowing through the connector switch.

In one aspect, the electrosurgical apparatus further comprises a flow control mechanism for controlling the flow rate of the at least one first fluid or the at least one second fluid through the fluid tube.

In one aspect, the electrosurgical apparatus is provided, wherein the at least one electrode is configured as a planar blade having a tapered distal point and a beveled edge such that the at least one porous electrode is suitable for electrosurgical cutting when energized and mechanical cutting when de-energized.

In one aspect, the electrosurgical apparatus further comprises a shaft made of an insulating material, the shaft disposed around the fluid tube.

In one aspect, the electrosurgical apparatus is provided, wherein the shaft and the fluid tube are configured to be flexible and the distal end of the shaft is configured to be grasped by forceps of a device to manipulate the orientation of the distal end of the shaft.

In another aspect of the present disclosure, an electrosurgical apparatus is provided comprising: a handle including an interior, a proximal end, and a distal end; first and second fluid tubes each including a proximal end and a distal end, the proximal end of each fluid tube disposed through the distal end of the handle into the interior of the handle; a first porous electrode coupled to the distal end of the first fluid tube; a second porous electrode coupled to the distal end of the second fluid tube; a y-connector disposed in the interior of the handle, the y-connector including a proximal end having a first fluid channel and a distal end having a second and third fluid channel, wherein the proximal end of the first fluid tube is coupled to the second fluid channel and the proximal end of the second fluid tube is coupled to the third fluid channel; and a connector switch disposed in the interior of the handle and coupled to the first fluid channel of the y-connector, the connector switch configured to receive at least one first fluid from a first fluid source and at least one second fluid from a second fluid source and, responsive to a user input, provide one of the at least one first fluid or the at least one second fluid to the first fluid channel of the y-connector, wherein the y-connector is configured to split fluid provided to the first channel and provide the fluid via the second channel to the first fluid tube and provide the fluid via the third channel to the second fluid tube, the first fluid tube configured to provide the fluid to the first porous electrode and the second fluid tube configured to provide the fluid to the second porous electrode, wherein the first and second porous electrodes each include a porous structure, the porous structure of the first electrode configured to allow fluid provided via the first fluid tube to flow through the porous structure of the first porous electrode and exit the first porous electrode, the porous structure of the second electrode configured to allow fluid provided via the second fluid tube to flow through the porous structure of the second porous electrode and exit the second porous electrode, wherein the first porous electrode is configured as an active electrode for receiving electrosurgical energy to be applied to patient tissue and the second porous electrode is configured as a return electrode for returning electrosurgical energy applied to the patient tissue.

In one aspect, the electrosurgical apparatus is provided, wherein the at least one first fluid is an electrically conducting fluid.

In one aspect, the electrosurgical apparatus is provided, wherein the electrically conducting fluid is saline.

In one aspect, the electrosurgical apparatus is provided, wherein the at least one second fluid is an inert gas.

In one aspect, the electrosurgical apparatus is provided, wherein when the inert gas is provided to the first and second electrodes and energy is applied across the first and second electrodes, plasma is generated to be applied to patient tissue.

In one aspect, the electrosurgical apparatus is provided, wherein the inert gas is helium.

In one aspect, the electrosurgical apparatus is provided wherein the connector switch is a 3-way fluid valve.

In one aspect, the electrosurgical apparatus is provided, wherein the connector switch is a microelectromechanical system valve.

In one aspect, the electrosurgical apparatus is provided, wherein the y-connectors includes an outer wall and an interior and the electrosurgical apparatus further comprises a first conductor disposed through the outer wall of the y-connector and into the interior of the first fluid tube and coupled to the first porous electrode and a second conductor disposed through outer wall of the y-connector and into the interior of the second fluid tube and coupled to the second porous electrode, the first and second conductors coupled to an energy source for providing electrosurgical energy across the first and second porous electrodes.

In one aspect, the electrosurgical apparatus is provided wherein the first and second fluid tubes are each made of a conductive material and coupled to an energy source for providing electrosurgical energy across the first and second porous electrodes.

In one aspect, the electrosurgical apparatus is provided, further comprising a circuit configured to change a state of the connector switch, wherein in a first state the connector switch is configured to enable the at least one first fluid to flow through the connector switch to be provided to the first and second porous electrodes and to block the at least one second fluid from flowing through the connector switch and in a second state the connector switch is configured to enable the at least one second fluid to flow through the connector switch to be provided to the first and second electrodes and to block the at least one first fluid from flowing through the connector switch.

In one aspect, the electrosurgical apparatus further comprises a flow control mechanism for controlling the flow rate of the at least one first fluid or the at least one second fluid through the first and second fluid tubes.

In one aspect, the electrosurgical apparatus further comprises a first shaft and a second shaft each made of an insulating material, the first shaft disposed around the first fluid tube and the second shaft disposed around the second fluid tube.

In one aspect, the electrosurgical apparatus is provided, wherein the first shaft, the second shaft, the first fluid tube, and the second fluid tube are configured to be flexible and the distal end of the first shaft and the distal end of the second shaft are each configured to be grasped by forceps of a device to manipulate the orientation of the distal ends of the first shaft and the second shaft.

In another aspect of the present disclosure, an electrosurgical generator is provided comprising: a receptacle configured to receive a connector of an electrosurgical apparatus; first and second pins, each coupled to the receptacle and configured to be electrically coupled with respective conductors disposed in the connector of the electrosurgical apparatus when the connector of the electrosurgical apparatus is received by the receptacle; a controller coupled to the first pin and configured to receive at least one signal via the first pin from the electrosurgical apparatus coupled to the receptacle; a radio-frequency (RF) energy source controllable by controller and coupled to the second pin, the RF energy source configured to generate electrosurgical energy and provide the electrosurgical energy to the second pin to be provided to the electrosurgical apparatus; a fluid tube including first and second ends, the first end coupled to the receptacle and configured to be coupled with a tube in the connector of the electrosurgical apparatus when the connector of the electrosurgical apparatus is received by the receptacle; a connector switch controllable by the controller and coupled to the second end of the fluid tube, the connector switch configured to receive at least one first fluid from a first fluid source and at least one second fluid from a second fluid source and, responsive to at least one signal received from the controller, provide one of the at least one first fluid or the at least one second fluid to the fluid tube to be provided to the electrosurgical apparatus.

In one aspect the electrosurgical generator further comprises a third pin coupled to the receptacle and the RF energy source, the third pin configured be electrically coupled with a conductor disposed in the connector of the electrosurgical apparatus and to provide a return path for electrosurgical energy provided to the electrosurgical apparatus via the second pin.

In one aspect the electrosurgical generator further comprises first and second fluid pumps and third and fourth fluid tubes, the first fluid pump coupled to the connector switch and the first fluid source via the third fluid tube and configured to pump the at least one first fluid from the first fluid source to the connector switch, the second fluid pump coupled to the connector switch and the second fluid source via the fourth fluid tube and configured to pump the at least one second fluid from the second fluid source to the connector switch.

In one aspect the electrosurgical generator is provided, wherein the controller is configured to control the first and second fluid pumps to control a flow rate of the first fluid or the second fluid.

In one aspect the electrosurgical generator is provided, wherein the controller is configured to control the first and second fluid pumps responsive to one or more signals received from the first pin.

In one aspect the electrosurgical generator is provided, wherein the controller is configured to control the connector switch to switch between a first state and a second state, in the first state, the connector switch is configured to enable the at least one first fluid to flow through the connector switch and into the fluid tube and connector switch blocks the at least one second fluid from flowing through the connector switch, and, in the second state, the connector switch is configured to enable the at least one second fluid to flow through the connector switch and into the fluid tube and connector switch blocks the at least one first fluid from flowing through the connector switch.

In one aspect the electrosurgical generator is provided, wherein the controller is configured to control the connector switch responsive to at least one signal received from the first pin.

In another aspect of the present disclosure, an electrosurgical apparatus is provided comprising: a handle including an interior, a proximal end, and a distal end; at least one fluid tube including a proximal end and a distal end, the proximal end of the at least one fluid tube disposed through the distal end of the handle into the interior of the handle; at least one porous electrode coupled to the distal end of the at least one fluid tube; and the at least one fluid tube configured to receive inert gas via the distal end of the at least one fluid tube and provide the inert gas to the at least one porous electrode, wherein the at least one porous electrode includes a porous structure configured to allow the inert gas provided via the at least one fluid tube to flow through the porous structure and exit the at least one porous electrode, wherein the at least one porous electrode is configured to receive and conduct electrosurgical energy from an energy source, such that when the at least one porous electrode is energized and inert gas is provided to the at least one porous electrode, plasma is generated and ejected from the at least one porous electrode.

In one aspect, the electrosurgical apparatus is provided, wherein the inert gas is helium.

In one aspect, the electrosurgical apparatus is provided, wherein the plasma is ejected as a diffuse plasma cloud.

In one aspect, the electrosurgical apparatus is provided, wherein the porous structure of the at least one porous electrode comprises a subset of the entire volume of the at least one porous electrode to control the geometry of the generated diffuse plasma cloud.

In one aspect, the electrosurgical apparatus is provided, wherein different regions of the porous structure are selectively configured with different levels of porosity to control the geometry of the generated diffuse plasma cloud.

It is to be appreciated that the various features shown and described are interchangeable, that is a feature shown in one embodiment may be incorporated into another embodiment.

While the disclosure has been shown and described with reference to certain preferred embodiments thereof, it will be understood by those skilled in the art that various changes in form and detail may be made therein without departing from the spirit and scope of the disclosure as defined by the appended claims.

Furthermore, although the foregoing text sets forth a detailed description of numerous embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘ ______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. An electrosurgical apparatus comprising:
a handle including an interior, a proximal end, and a distal end;
a fluid tube including a proximal end and a distal end, the proximal end of the fluid tube disposed through the distal end of the handle into the interior of the handle;
at least one porous electrode coupled to the distal end of the fluid tube; and
a connector switch disposed in the interior of the handle and coupled to the proximal end of the fluid tube, the connector switch configured to receive at least one first fluid from a first fluid source and at least one second fluid from a second fluid source and, responsive to a user input, provide one of the at least one first fluid or the at least one second fluid to the fluid tube, the fluid tube configured to provide the fluid to the at least one porous electrode,
wherein the at least one porous electrode includes a porous structure configured to allow fluid provided via the fluid tube to flow through the porous structure and exit the at least one porous electrode,
wherein the at least one porous electrode is configured to receive and conduct electrosurgical energy from an energy source.

2. The electrosurgical apparatus of claim 1, wherein the at least one first fluid is an electrically conducting fluid.

3. The electrosurgical apparatus of clam 2, wherein the electrically conducting fluid is saline.

4. The electrosurgical apparatus of claim 1, wherein the at least one second fluid is an inert gas.

5. The electrosurgical apparatus of claim 4, wherein when the at least one porous electrode is energized and the inert gas is provided to the at least one porous electrode, plasma is generated and ejected from the at least one porous electrode.

6. The electrosurgical apparatus of claim 5, wherein the plasma is ejected as a diffuse plasma cloud.

7. The electrosurgical apparatus of claim 6, wherein the porous structure of the at least one porous electrode comprises a subset of the entire volume of the at least one porous electrode to control the geometry of the generated diffuse plasma cloud.

8. The electrosurgical apparatus of claim 6, wherein different regions of the porous structure are selectively configured with different levels of porosity to control the geometry of the generated diffuse plasma cloud.

9. The electrosurgical apparatus of claim 4, wherein the inert gas is helium.

10. The electrosurgical apparatus of claim 1, wherein the connector switch is a 3-way fluid valve.

11. The electrosurgical apparatus of claim 1, wherein the connector switch is a microelectromechanical system valve.

12. The electrosurgical apparatus of claim 1, wherein the fluid tube includes an outer wall and an interior and the electrosurgical apparatus further comprises a conductor disposed through the outer wall and into the interior of the fluid tube and coupled to the at least one porous electrode, the conductor configured to receive electrosurgical energy from the energy source and provide the electrosurgical energy to the at least one porous electrode.

13. The electrosurgical apparatus of claim 1, wherein the fluid tube is made of a conductive material and configured to receive electrosurgical energy from the energy source and provide the electrosurgical energy to the at least one porous electrode.

14. The electrosurgical apparatus of claim 1, further comprising a circuit configured to change a state of the connector switch, wherein in a first state the connector switch is configured to enable the at least one first fluid to be provided to the at least one porous electrode via the fluid tube and to block the at least one second fluid from flowing through the connector switch and in a second state the connector switch is configured to enable the at least one second fluid to be provided to the at least one porous electrode via the fluid tube and to block the at least one first fluid from flowing through the connector switch.

15. The electrosurgical apparatus of claim 1, further comprising a flow control mechanism for controlling the flow rate of the at least one first fluid or the at least one second fluid through the fluid tube.

16. The electrosurgical apparatus of claim 1, wherein the at least one electrode is configured as a planar blade having a tapered distal point and a beveled edge such that the at least one porous electrode is suitable for electrosurgical cutting when energized and mechanical cutting when de-energized.

17. The electrosurgical apparatus of claim 1, further comprising a shaft made of an insulating material, the shaft disposed around the fluid tube.

18. The electrosurgical apparatus of claim 17, wherein the shaft and the fluid tube are configured to be flexible and the distal end of the shaft is configured to be grasped by forceps of a device to manipulate the orientation of the distal end of the shaft.

19. An electrosurgical apparatus comprising:
a handle including an interior, a proximal end, and a distal end;
first and second fluid tubes each including a proximal end and a distal end, the proximal end of each fluid tube disposed through the distal end of the handle into the interior of the handle;
a first porous electrode coupled to the distal end of the first fluid tube;
a second porous electrode coupled to the distal end of the second fluid tube;
a y-connector disposed in the interior of the handle, the y-connector including a proximal end having a first fluid channel and a distal end having a second and third fluid channel, wherein the proximal end of the first fluid tube is coupled to the second fluid channel and the proximal end of the second fluid tube is coupled to the third fluid channel; and
a connector switch disposed in the interior of the handle and coupled to the first fluid channel of the y-connector, the connector switch configured to receive at least one first fluid from a first fluid source and at least one second fluid from a second fluid source and, responsive to a user input, provide one of the at least one first fluid or the at least one second fluid to the first fluid channel of the y-connector,
wherein the y-connector is configured to split fluid provided to the first channel and provide the fluid via the second channel to the first fluid tube and provide the fluid via the third channel to the second fluid tube, the first fluid tube configured to provide the fluid to the first porous electrode and the second fluid tube configured to provide the fluid to the second porous electrode,
wherein the first and second porous electrodes each include a porous structure, the porous structure of the first electrode configured to allow fluid provided via the first fluid tube to flow through the porous structure of the first porous electrode and exit the first porous electrode, the porous structure of the second electrode configured to allow fluid provided via the second fluid tube to flow through the porous structure of the second porous electrode and exit the second porous electrode,
wherein the first porous electrode is configured as an active electrode for receiving electrosurgical energy to be applied to patient tissue and the second porous electrode is configured as a return electrode for returning electrosurgical energy applied to the patient tissue.

20. The electrosurgical apparatus of claim 19, wherein the at least one first fluid is an electrically conducting fluid.

21. The electrosurgical apparatus of clam 20, wherein the electrically conducting fluid is saline.

22. The electrosurgical apparatus of claim 19, wherein the at least one second fluid is an inert gas.

23. The electrosurgical apparatus of claim 22, wherein when the inert gas is provided to the first and second electrodes and energy is applied across the first and second electrodes, plasma is generated to be applied to patient tissue.

24. The electrosurgical apparatus of claim 22, wherein the inert gas is helium.

25. The electrosurgical apparatus of claim 19, wherein the connector switch is a 3-way fluid valve.

26. The electrosurgical apparatus of claim 19, wherein the connector switch is a microelectromechanical system valve.

27. The electrosurgical apparatus of claim 19, wherein the y-connectors includes an outer wall and an interior and the electrosurgical apparatus further comprises a first conductor disposed through the outer wall of the y-connector and into the interior of the first fluid tube and coupled to the first porous electrode and a second conductor disposed through outer wall of the y-connector and into the interior of the second fluid tube and coupled to the second porous electrode, the first and second conductors coupled to an energy source for providing electrosurgical energy across the first and second porous electrodes.

28. The electrosurgical apparatus of claim 19, wherein the first and second fluid tubes are each made of a conductive material and coupled to an energy source for providing electrosurgical energy across the first and second porous electrodes.

29. The electrosurgical apparatus of claim 19, further comprising a circuit configured to change a state of the connector switch, wherein in a first state the connector switch is configured to enable the at least one first fluid to flow through the connector switch to be provided to the first and second porous electrodes and to block the at least one second fluid from flowing through the connector switch and in a second state the connector switch is configured to enable the at least one second fluid to flow through the connector switch to be provided to the first and second electrodes and to block the at least one first fluid from flowing through the connector switch.

30. The electrosurgical apparatus of claim 19, further comprising a flow control mechanism for controlling the flow rate of the at least one first fluid or the at least one second fluid through the first and second fluid tubes.

31. The electrosurgical apparatus of claim 19, further comprising a first shaft and a second shaft each made of an insulating material, the first shaft disposed around the first fluid tube and the second shaft disposed around the second fluid tube.

32. The electrosurgical apparatus of claim 31, wherein the first shaft, the second shaft, the first fluid tube, and the second fluid tube are configured to be flexible and the distal end of the first shaft and the distal end of the second shaft are each configured to be grasped by forceps of a device to manipulate the orientation of the distal ends of the first shaft and the second shaft.

33. An electrosurgical generator comprising:
a receptacle configured to receive a connector of an electrosurgical apparatus;
first and second pins, each coupled to the receptacle and configured to be electrically coupled with respective conductors disposed in the connector of the electrosurgical apparatus when the connector of the electrosurgical apparatus is received by the receptacle;
a controller coupled to the first pin and configured to receive at least one signal via the first pin from the electrosurgical apparatus coupled to the receptacle;
a radio-frequency (RF) energy source controllable by controller and coupled to the second pin, the RF energy source configured to generate electrosurgical energy and provide the electrosurgical energy to the second pin to be provided to the electrosurgical apparatus;
a fluid tube including first and second ends, the first end coupled to the receptacle and configured to be coupled with a tube in the connector of the electrosurgical apparatus when the connector of the electrosurgical apparatus is received by the receptacle;
a connector switch controllable by the controller and coupled to the second end of the fluid tube, the connector switch configured to receive at least one first fluid from a first fluid source and at least one second fluid from a second fluid source and, responsive to at least one signal received from the controller, provide one of the at least one first fluid or the at least one second fluid to the fluid tube to be provided to the electrosurgical apparatus.

34. The electrosurgical generator of claim 33, further comprising a third pin coupled to the receptacle and the RF energy source, the third pin configured be electrically coupled with a conductor disposed in the connector of the electro surgical apparatus and to provide a return path for electrosurgical energy provided to the electrosurgical apparatus via the second pin.

35. The electrosurgical generator of claim 33, further comprising first and second fluid pumps and third and fourth fluid tubes, the first fluid pump coupled to the connector switch and the first fluid source via the third fluid tube and configured to pump the at least one first fluid from the first fluid source to the connector switch, the second fluid pump coupled to the connector switch and the second fluid source via the fourth fluid tube and configured to pump the at least one second fluid from the second fluid source to the connector switch.

36. The electrosurgical generator of claim 35, wherein the controller is configured to control the first and second fluid pumps to control a flow rate of the first fluid or the second fluid.

37. The electrosurgical generator of claim 36, wherein the controller is configured to control the first and second fluid pumps responsive to one or more signals received from the first pin.

38. The electrosurgical generator of claim 33, wherein the controller is configured to control the connector switch to switch between a first state and a second state, in the first state, the connector switch is configured to enable the at least one first fluid to flow through the connector switch and into the fluid tube and connector switch blocks the at least one second fluid from flowing through the connector switch, and, in the second state, the connector switch is configured to enable the at least one second fluid to flow through the connector switch and into the fluid tube and connector switch blocks the at least one first fluid from flowing through the connector switch.

39. The electrosurgical generator of claim 38, wherein the controller is configured to control the connector switch responsive to at least one signal received from the first pin.

40. An electrosurgical apparatus comprising:
a handle including an interior, a proximal end, and a distal end;
at least one fluid tube including a proximal end and a distal end, the proximal end of the at least one fluid tube disposed through the distal end of the handle into the interior of the handle;
at least one porous electrode coupled to the distal end of the at least one fluid tube; and
the at least one fluid tube configured to receive inert gas via the distal end of the at least one fluid tube and provide the inert gas to the at least one porous electrode,
wherein the at least one porous electrode includes a porous structure configured to allow the inert gas provided via the at least one fluid tube to flow through the porous structure and exit the at least one porous electrode,
wherein the at least one porous electrode is configured to receive and conduct electrosurgical energy from an energy source, such that when the at least one porous electrode is energized and inert gas is provided to the at least one porous electrode, plasma is generated and ejected from the at least one porous electrode.

41. The electrosurgical apparatus of 40, wherein the inert gas is helium.

42. The electrosurgical apparatus of claim 40, wherein the plasma is ejected as a diffuse plasma cloud.

43. The electrosurgical apparatus of claim 42, wherein the porous structure of the at least one porous electrode comprises a subset of the entire volume of the at least one porous electrode to control the geometry of the generated diffuse plasma cloud.

44. The electrosurgical apparatus of claim 42, wherein different regions of the porous structure are selectively configured with different levels of porosity to control the geometry of the generated diffuse plasma cloud.
